(19) United States
(12) Patent Application Publication
(10) Pub. No.: US 2026/0263508 A1
(43) Pub. Date: Sep. 10, 2026

(54) POLYGLUTAMIC ACID COMPOSITIONS AND METHODS OF USING

(71) Applicant: Bonafide Health, LLC, Harrison, NY (US)

(72) Inventors: James R. Komorowski, Trumbull, CT (US); Devon Bernsley, New York, NY (US)

(21) Appl. No.: 19/659,892

(22) Filed: Apr. 27, 2026

Related U.S. Application Data
(63) Continuation-in-part of application No. 19/242,073, filed on Jun. 18, 2025.
Application No. 19/242,073 is a continuation of application No. 18/177,720, filed on Mar. 2, 2023, now Pat. No. 12,357,655.
(60) Provisional application No. 63/315,964, filed on Mar. 2, 2022.

(51) Int. Cl. A61K 31/785; A61K 9/00; A61K 47/10; A61K 47/14; A61K 47/26

(52) U.S. Cl. CPC A61K 31/785; A61K 9/0034; A61K 47/10; A61K 47/14; A61K 47/26

(57) ABSTRACT

Disclosed herein are compositions comprising one or more polyglutamic acid compounds. The compositions are for treating, ameliorating, preventing, or reducing the symptoms of vaginal dryness and the symptoms associated with menopause. Also described herein are suppository formulations and intravaginal ring formulations of the compositions.

### CROSS-REFERENCE TO RELATED APPLICATIONS

This application is a continuation-in-part of U.S. application Ser. No. 19/242,073, filed Jun. 18, 2025, which is a continuation of U.S. application Ser. No. 18/177,720, filed Mar. 2, 2023, granted as U.S. Pat. No. 12,357,655, which claims priority to U.S. Provisional Application No. 63/315,964, filed Mar. 2, 2022, the disclosures of each of which is incorporated herein by reference in their entirety.

### BACKGROUND

Vaginal dryness is a common condition that can affect women of all ages. However, vaginal dryness is most common in women following menopause. In postmenopausal women, vaginal dryness may be associated with declining estrogen levels. Topical estrogen therapy has been the most common treatment for vaginal dryness but this therapy carries risks such as vaginal bleeding and increased risk of uterine cancer. An alternative therapy for patients unwilling or unable to consider an estrogen therapy is hyaluronic acid. Yet, it also has adverse effect including vulvovaginal candidiasis and bacterial vaginosis. As such, the prior art methods and compositions for treatment of vaginal dryness are insufficient and solutions are provided herein to help alleviate vaginal dryness.

### SUMMARY

Embodiments of the present disclosure relate to novel compositions containing one or more polyglutamic acid compounds and their use in the treatment and/or amelioration of the symptoms of vaginal dryness and the symptoms associated with menopause.

These and other features, aspects, and advantages of the present embodiments will become understood with reference to the following description and appended claims.

### BRIEF DESCRIPTION OF THE DRAWINGS

FIG. 1 shows exemplary intravaginal ring shapes of the disclosure contained herein, e.g., circular shape, Y-shape, and M-shape, from left to right.

FIGS. 2A-FIG. 2E show exemplary intravaginal ring formulations.

FIG. 3A shows mean cumulative release percentage of the two polyglutamic acid compounds from three intravaginal matrix ring formulations described herein.

FIG. 3B shows the in-vitro elution of two polyglutamic acid compounds from four intravaginal matrix ring formulations described herein.

FIG. 4A shows the mean daily release amount of two polyglutamic acid compounds from an intravaginal matrix ring formulation described herein.

FIG. 4B shows the mean daily release amount of two polyglutamic acid compounds from four intravaginal matrix ring formulation described herein.

FIG. 4C shows the mean cumulative release percentage of two polyglutamic acid compounds from four intravaginal matrix ring formulation described herein.

### DETAILED DESCRIPTION

Described herein are compositions comprising one or more polyglutamic acid compounds and a pharmaceutically acceptable carrier. Each polyglutamic acid compound can be prepared by reacting a glutamic ester monomer with a suitable initiator. The molecular weight of each polyglutamic acid compound is determined by the number of the glutamic acid monomers that have been linked, corresponding to an n value set forth below.

The structure of a polyglutamic acid compound is depicted below:

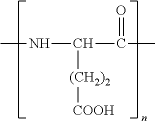

N refers to a chain length of a polyglutamic acid compound and can be between 300 and 30,000. The molecular weight of a polyglutamic acid compound of the disclosure can be between about 50 and 4,000 kDa. The composition described herein can contain one or more polyglutamic acid compounds that can have different polymer chain lengths (i.e., different n values).

For example, and without limitation, a composition can be comprised of a first polyglutamic acid compound, wherein the first polyglutamic acid compound has an n value of between about 300 and 500; a second polyglutamic acid compound, wherein the second polyglutamic acid compound has an n value between about 1,000 and 10,000; and a third polyglutamic acid compound, wherein the third polyglutamic acid compound has an n value between about 20,000 and 30,000.

The number of polyglutamic acid compounds having different n values in a composition is not particularly limited. In some embodiments, a composition can comprise one, two, three, four, five, six, or more different polyglutamic acid compounds that have different n values.

Without being bound to any particular theory, the compositions containing one or more polyglutamic acid compounds of the instant disclosure produce unexpectedly superior results due to the compounds' ability to penetrate varying layers of vaginal epithelial tissue depending upon the particular ratio of polyglutamic acid compounds having different n values. For example, a composition can comprise a first polyglutamic acid compound and a second polyglutamic acid compound, wherein the first polyglutamic acid compound has an n value that is less than the second polyglutamic acid compound. It is believed that a polyglutamic acid compound having a smaller n value is able to penetrate into deeper layers of the vaginal epithelium than a polyglutamic acid compound with a larger n value. And it is further believed that a second polyglutamic acid compound having an n value greater than a first polyglutamic acid compound will not penetrate into deeper epithelial layers, but rather, the second polyglutamic acid compound will stay closer to the surface than the first polyglutamic acid compound.

Certain embodiments comprise a first polyglutamic acid compound, a second polyglutamic acid compound, and a third polyglutamic acid compound. In certain embodiments, the first polyglutamic acid compound is a polyglutamic acid compound comprising a greater number of glutamic acid monomers than the second polyglutamic acid compound. The second polyglutamic acid compound comprises a greater number of glutamic acid monomers than the third polyglutamic acid compound. In certain embodiments, the first polyglutamic acid compound is configured to penetrate into the stratus corneum layer of a subject's vaginal wall, the second polyglutamic acid compound is configured to penetrate into the stratum granulosum layer of a subject's vaginal wall, and the third polyglutamic acid compound is configured to penetrate into the stratum spinosum layer of a subject's vaginal wall. In some embodiments, one or more of the polyglutamic acid compounds can have an n value large enough such that it does not penetrate any skin layers, but stays on the skin surface, and can act as a lubricant.

The amount of each polyglutamic acid compound in any of the compositions described herein is not particularly limited, and may be about 10 μg to about 10 g. For example, the amount can be 10 μg, 15 μg, 20 μg, 25 μg, 30 μg, 35 μg, 40 μg, 45 μg, 50 μg, 55 μg, 60 μg, 65 μg, 70 μg, 75 μg, 80 μg, 85 μg, 90 μg, 95 μg, 100 μg, 125 μg, 150 μg, 175 μg, 200 μg, 225 μg, 250 μg, 275 μg, 300 μg, 325 μg, 350 μg, 375 μg, 400 μg, 425 μg, 450 μg, 475 μg, 500 μg, 525 μg, 575 μg, 600 μg, 625 μg, 650 μg, 675 μg, 700 μg, 725 μg, 750 μg, 775 μg, 800 μg, 825 μg, 850 μg, 875 μg, 900 μg, 925 μg, 950 μg, 975 μg, 1000 μg, 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 125 mg, 150 mg, 175 mg, 200 mg, 225 mg, 250 mg, 275 mg, 300 mg, 325 mg, 350 mg, 375 mg, 400 mg, 425 mg, 450 mg, 475 mg, 500 mg, 525 mg, 575 mg, 600 mg, 625 mg, 650 mg, 675 mg, 700 mg, 725 mg, 750 mg, 775 mg, 800 mg, 825 mg, 850 mg, 875 mg, 900 mg, 925 mg, 950 mg, 975 mg, 1000 mg, 1.25 g, 1.5 g, 1.75 g, 2.0 g, 2.25 g, 2.5 g, 2.75 g, 3.0 g, 3.25 g, 3.5 g, 3.5 g, 3.75 g, 4.0 g, 4.25 g, 4.5 g, 4.75 g, 5.0 g, 5.25 g, 5.5 g, 5.75 g, 6.0 g, 6.25 g, 6.5 g, 6.75 g, 7.0 g, 7.25 g, 7.5 g, 7.75 g, 8.0 g, 8.25 g, 8.5 g, 8.75 g, 9.0 g, 8.25 g, 9.5 g, 9.75 g, 10 g, or any amount therebetween.

In certain embodiments, one or more polyglutamic acid compounds in a composition described herein have molecular weights ranging from about 50 kDa to about 2000 kDa, from about 100 kDa to about 2000 kDa, or from about 100 kDa to about 700 kDa. In certain embodiments, a composition described herein comprises two or more polyglutamic acid compounds. In certain embodiments, a composition described herein comprises two polyglutamic acid compounds. The ratio of a first polyglutamic acid compound to a second polyglutamic acid compound can range from about 10:1 to about 1:10, from about 5:1 to 1:5, from about 3:1 to about 1:3, or from about 3:1 to about 1:1 by their weight percentages in the composition. For example, the ratio of a first polyglutamic acid compound to a second polyglutamic acid compound can be about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, or any values in between. The weight percentage (% w/w) of each polyglutamic acid compound in any of the compositions described herein can range from about 0.01 wt % to about 90 wt %. For example, it can be about 0.01 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, 66 wt %, 67 wt %, 68 wt %, 69 wt %, 70 wt %, 71 wt %, 72 wt %, 73 wt %, 74 wt %, 75 wt %, 76 wt %, 77 wt %, 78 wt %, 79 wt %, 80 wt %, 81 wt %, 82 wt %, 83 wt %, 84 wt %, 85 wt %, 86 wt %, 87 wt %, 88 wt %, 89 wt %, 90 wt %, or any value therebetween. In certain embodiments, a composition described herein comprises about 0.01-90 wt % of polyglutamic acid compounds in total. For example, a composition described herein can comprise about 0.01 wt %, 0.1 wt %, 1 wt %, 10 wt %, 20 wt %, 25 wt %, 30 wt %, 40 wt %, 50 wt %, 60 wt %, 70 wt %, 80 wt %, 90 wt % of the total amount of polyglutamic acid compounds. In certain embodiments, a composition described herein comprises two polyglutamic acid compounds having molecular weights selected from about 100 kDa, 700 kDa, and 2000 kDa. In certain embodiments, a composition described herein comprises two polyglutamic acid compounds with a first polyglutamic acid compound having a molecular weight of about 100 kDa and a second polyglutamic acid compound having a molecular weight of about 700 kDa. Without being bound by any theory, it is believed that a polyglutamic acid compound having a molecular weight greater than about 700 kDa does not pass through the stratum cornuem and thus enhances moisture-binding on the surface and provides lubrication and a polyglutamic acid compound having a molecular weight of about 100 kDa penetrates the epidermis, stays between the epidermis and the dermis, and provides hydration due to its water absorbing capacity. The ratios of a first polyglutamic acid compound having a molecular weight of about 100 kDa and a second polyglutamic acid compound having a molecular weight of about 700 kDa are set forth above. In certain embodiments, a first polyglutamic acid compound having a molecular weight of about 100 kDa and a second polyglutamic acid compound having a molecular weight of about 700 kDa are provided in a ratio of about 3:1 by their weight percentages in a composition.

The compositions disclosed herein can be used to treat, prevent, or alleviate vaginal dryness, vaginal itching, and/or vaginal burning. In some embodiments, the compositions described herein can be used to maintain healthy levels of vaginal moisture. In some embodiments, the compositions described herein can be used to accelerate the rate of wound healing, prevent urinary tract infections, and/or maintain pH values in a vaginal tissue. In certain embodiments, a composition as described herein is administered to a subject to avoid painful intercourse. In certain embodiments, a composition as described herein is administered to a subject to rejuvenate a vaginal tissue.

In certain embodiments, a composition can be administered to a subject's vaginal tissue to treat, prevent, ameliorate, and/or reduce the symptoms of vaginal dryness. In certain embodiments, a composition can be administered to a subject's vaginal tissue to maintain a healthy level of vaginal moisture, characterized by slight, natural wetness to protect against vaginal irritation and infection as would be experienced by a healthy individual as that would be understood by the artisan of skill in the art. In certain embodiments, a composition can be administered to a subject's vaginal tissue to treat, prevent, and/or ameliorate the symptoms of vaginal dryness due to menopause. In some embodiments, a composition can be administered to a subject's vaginal tissue to treat, prevent, and/or ameliorate one or more symptoms of menopause. In certain embodiments, a composition can be administered to a subject's vaginal tissue to reduce the pain associated with sexual activity, including sexual intercourse. In certain embodiments, a composition can be administered to a subject's vaginal tissue to treat, prevent, and/or ameliorate vaginal itching. In certain embodiments, a composition can be administered to a subject's vaginal tissue to treat, prevent, and/or ameliorate vaginal burning. In certain embodiments, a composition can be used for one or more of the foregoing.

In some embodiments, the compositions disclosed herein are in the form of pharmaceutically effective salts. The phrase “pharmaceutically acceptable salt(s),” is art recognized and, as used herein includes, but is not limited to, salts of acidic or basic groups that may be present in the compositions disclosed herein. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including but not limited to sulfuric, citric, maleic, acetic, oxalic, hydrochloride, hydrobromide, hydroiodide, nitrate, sulfate, bisulfate, phosphate, acid phosphate, isonicotinate, acetate, lactate, salicylate, citrate, acid citrate, tartrate, oleate, tannate, pantothenate, bitartrate, ascorbate, succinate, maleate, gentisinate, fumarate, gluconate, glycerate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds present in the compositions disclosed herein that include an amino moiety also can form pharmaceutically acceptable salts with various amino acids, in addition to the acids mentioned above. Compounds present in the compositions disclosed herein that are acidic in nature are capable of forming base salts with various pharmacologically acceptable cations. Non limiting examples of such salts include alkali metal or alkaline earth metal salts and, particularly, calcium, magnesium, sodium lithium, zinc, potassium, silicon, phosphorus, and iron salts.

The phrase “pharmaceutically acceptable carrier” is art-recognized and includes a pharmaceutically acceptable material, composition or vehicle, suitable for administering compounds of the present invention to mammals. The carriers include liquid or solid filler, diluent, excipient, solvent or encapsulating material, involved in carrying or transporting the subject agent from one organ, or portion of the body, to another organ, or portion of the body.

Each carrier must be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose and its derivatives, such as sodium carboxymethyl cellulose, ethyl cellulose, and cellulose acetate; powdered tragacanth; malt; gelatin; talc; excipients, such as cocoa butter and suppository waxes; oils, such as peanut oil, cottonseed oil, safflower oil, sesame oil, olive oil, corn oil, and soybean oil; glycols, such as propylene glycol; polyols, such as glycerol, glycerides, sorbitol, mannitol, and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. In one embodiment, the pharmaceutically acceptable carrier is suitable for intravenous administration. In another embodiment, the pharmaceutically acceptable carrier is suitable for locoregional injection.

The term “pharmaceutically acceptable esters” refers to the relatively non-toxic, esterified products of the compounds of the present invention. These esters can be prepared in situ during the final isolation and purification of the compounds, or by separately reacting the purified compound in its free acid form or hydroxyl with a suitable esterifying agent. Carboxylic acids can be converted into esters via treatment with an alcohol in the presence of a catalyst. Hydroxyls can be converted into esters via treatment with an esterifying agent such as alkanoyl halides. The term also includes lower hydrocarbon groups capable of being solvated under physiological conditions, e.g., alkyl esters, methyl, ethyl, and propyl esters.

As used herein, the singular forms “a”, “an” and “the” include plural referents unless the context clearly dictates otherwise. The disclosure of a “ratio” of compounds and compositions corresponds to a ratio provided in terms of weight mass of the components present in the ratio. To provide a more concise description, some of the quantitative expressions given herein are not qualified with the term “about.” It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value. As used herein, the term “subject” refers to mammals, including humans.

The dosage form of compositions described herein is not particularly limited. In certain embodiments, a composition described herein is formulated as a topical, such as a cream or an ointment. In certain embodiments, a composition described herein is formulated as a gel. In certain embodiments, a composition described herein is formulated as a suppository. In certain embodiments, a composition described herein is formulated as an intravaginal ring. In certain embodiments, a composition described herein is formulated as a tablet. In certain embodiments, a composition described herein is formulated as a capsule. In certain embodiments, a composition described herein is formulated as a foam. In certain embodiments, a composition described herein is formulated as a pessary. In certain embodiments, a composition described herein is formulated as a film. In certain embodiments, a composition described herein is formulated as a patch. In certain embodiments, a composition described herein is formulated as a microsphere. In certain embodiments, a composition described herein is formulated as a nanoparticle. In certain embodiments, a composition described herein is formulated as a microparticle. In certain embodiments, a composition described herein is formulated as a pellet. In certain embodiments, the dosage form of a composition described herein is mucoadhesive. In certain embodiments, the dosage form of a composition described herein is a material capable of thermogelling (i.e., forming a gel when inside the vagina). In certain embodiments, a composition described herein is formulated as a thermosensitive dosage form. In certain embodiments, a composition described herein is formulated as a pH-sensitive dosage form.

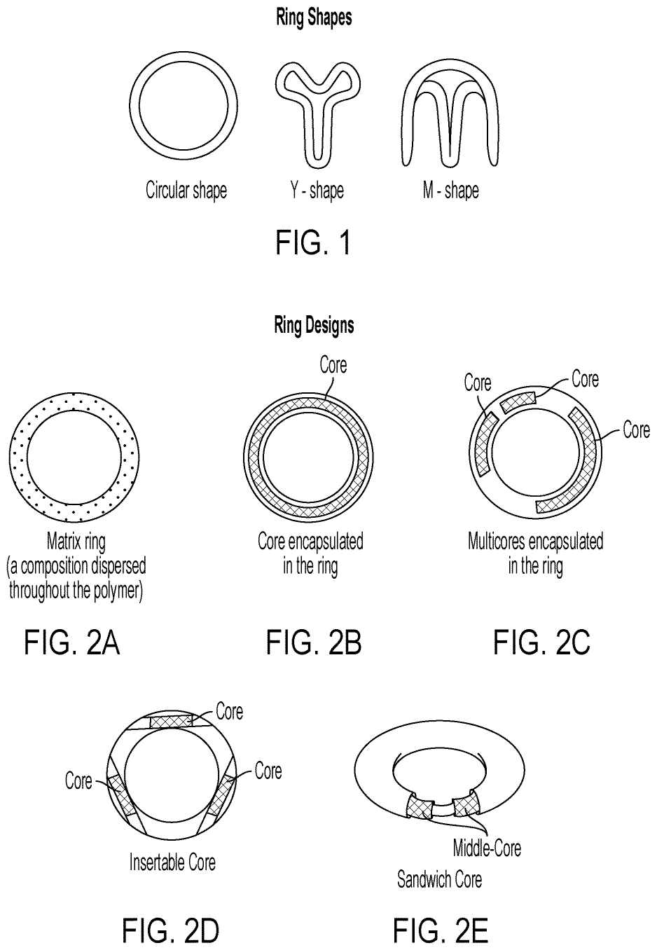

The shapes of dosage forms described herein are not particularly limited. In certain embodiments, a suppository described herein can have a bullet shape, a round oval shape, an elongated oval shape, a toroidal shape, a cylindrical shape, a teardrop shape, a tampon shape or the like. In certain embodiments, a tablet described herein can have a flat-faced shape, a round shape, or a cylindrical shape. In certain embodiments, an intravaginal ring described herein has a circular (or torus) shape, a Y shape, or a M shape as shown in FIG. 1.

In certain embodiments, a composition described herein can be in a sustained-release formulation. Sustained release refers to any one of extended-release dosage forms. The following terms may be considered to be substantially equivalent to sustained release, for the purposes of the present disclosure: continuous release, controlled release, delayed release, depot, gradual release, long term release, programmed release, prolonged release, programmed release, proportionate release, protracted release, repository, retarded release, slow release, spaced release, time coat, time release, delayed action, extended action, layered time action, long acting, prolonged action, sustained action medications, extended release, and release in terms of pH level in the vagina. As used herein, “release in terms of pH level in the vagina” is based on the common knowledge in the field that vaginal pH has an ideal range of 3.8 to 4.5 and can be higher than 4.5 before menstruation and after menopause and certain carriers such as pH-responsive hydrogels can release their payload at specific pH levels.

In certain embodiments, a sustained-release formulation can be obtained by using sufficient amounts of excipients to produce a sustained-release effect. To control release rates of the composition, excipients can be mixed with a composition described herein, form a membrane coating the composition, form a device where the composition can be dispersed or impregnated, or any combinations thereof. Excipients can include, but are not limited to polymers, fatty acids, fatty/oleaginous bases, viscosity modifiers, preservatives, silicon dioxide, humectants, and solvents. The polymers can be from plant sources, from animal sources, semisynthetic, or synthetic. Examples of polymers include, but are not limited to, gelatin (e.g., glycerinated gelatin), natural sugars (e.g., raw sugar and lactose), alginates, carrageenan, chitosan, gellan gum, lecithin, mucilage, plant gums, pectin, pectin derivatives, algal polysaccharides, glucomannan, agar, lignin, guar gum, locust bean gum, acacia gum, xanthan gum, carrageenan gum, karaya gum, tragacanth gum, ghatti gum, starches (e.g., corn starch and amylose), dextran, polyvinyl pyrrolidone, polyvinyl acetate, gum arabic, alginic acid, tylose, talcum lycopodium, silica gel (e.g., colloidal), pharmaceutically acceptable monovalent or multivalent alcohols and polyglycols such as polyethylene glycols (PEGs) of various molecular weights (e.g., PEG 400, PEG 1000, PEG 4000, and PEG 6000) and derivatives thereof, polysorbates (e.g., Tween 61 and Tween 80), cellulose, cellulose derivatives (e.g., microcrystalline cellulose, cellulose ethers, methoxypropyl cellulose, methyl cellulose, hydroxypropyl methyl cellulose, hydroxypropyl methyl cellulose phthalate, cross-linked sodiumcarboxymethylcellulose,
cross-linked hydroxypropylcellulose, high-molecular weight hydroxymethylpropylcellulose, carboxymethyl-cellulose, low-molecular weight hydroxypropylmethycellulose, hydroxyethylcellulose, medium-viscosity hydroxypropylcellulose, hydroxypropylmethylcellulose, sodium carboxymethylcellulose, alkyl celluloses, ethyl cellulose, cellulose acetate, cellulose propionate, cellulose acetatepropionate, cellulose acetate butyrate, cellulose triacetate, and hydroxypropyl cellulose), poloxamers, sodium lauryl sulfate, polyacrylates, polyinylpyrrolidone, polylactic acid, polyethylene glycol, polyurethane, polyetheramide, polyether, alkyds, polyhydroxyurethane, and poly(ethylene-co-vinyl acetate) (“EVA”). Fatty acids include saturated fatty acids (e.g., stearates such as magnesium stearate), glycerol esters and polyglycerol esters of saturated fatty acids and their mixtures, magnesium salts of fatty acids, calcium salts of fatty acids, aluminum salts of fatty acids, fatty acids esterified with sorbitol anhydrides (i.e., polysorbates such as Tween 61 and Tween 80), esters of aliphatic saturated or unsaturated fatty acids with monovalent aliphatic alcohols, multivalent alcohols (e.g., glycols, glycerol, diethylene glycol, pentacrythritol, sorbitol, and mannitol), esterified with monovalent aliphatic alcohols, esterified with multivalent alcohols, esters of citric acid with primary alcohols, acetic acid, urea, benzyl benzoate, dioxolanes, glycerofonnals, tetrahydrofurfuryl alcohol, polyglycol ethers, dimethylacetamide, lactamides, lactates, ethylcarbonates, silicones, calcium carbonate, sodium carbonate, calcium phosphate, sodium phosphate, magnesium carbonate and the like, and polycarboxylic acids. Fatty/oleaginous bases include coconut butter (theobroma oil), hard fats (e.g., Witepsol and Suppocire), hydrogenated vegetable oils (e.g., hydrogenated palm kernel oil and hydrogenated coconut oi), synthetic glycerides (e.g., glyceryl monostearate and glyceryl monopalmitate), and vegetable oils (e.g., peanut oil, castor oil, olive oil, sesame oil, cottonseed oil, corn oil, wheat germ oil, sunflower seed oil, and cod liver oil). Examples of viscosity modifiers include but are not limited to beewax, cetyl alcohol, stearyl alcohol, and microcrystalline wax. Examples of preservatives include but are not limited to methylparaben, propylparaben, butylate hydroxytoluene (BHT), and butylate hydroxyanisole (BHA). As used herein, EVA refers to a copolymer with varying compositions of ethylene and vinyl acetate. Examples of EVA include, but are not limited to, EVA 40, EVA 33, EVA 28, EVA 18, EVA 12, and EVA 9. The number refers to the vinyl acetate content percentage of EVA. For example, EVA 28 contains 28% vinyl acetate and 72% ethylene and EVA 33 contains 33% vinyl acetate and 67% ethylene. The higher of the vinyl acetate content, the softer and more flexible of the EVA.

In certain embodiments, the sustained-release formulation can be obtained by using certain systems. Examples of the systems include, but are not limited to, reservoir systems with rate-controlling membranes such as encapsulation (including micro-encapsulation and macro-encapsulation) and membrane systems; reservoir systems without rate-controlling membranes such as hollow fibers, ultra macroporous cellulose triacetate, and porous polymeric substrates and foams; monolithic systems including systems physically dissolved in non-porous, polymeric or elastomeric matrices (e.g., non-erodible, erodible, environmental agent ingression, and degradable) and materials physically dispersed in non-porous, polymeric, or elastomeric matrices (e.g., non-erodible, erodible, environmental agent ingression, and degradable); laminated structures including reservoir layers chemically similar or dissimilar to outer control layers; chemical erosion of polymer matrices (e.g., heterogeneous or homogeneous erosion), biological erosion of a polymer matrix (e.g., heterogeneous or homogeneous), hydrogels, and the like.

In certain embodiments, a suppository formulation comprises one or two polyglutamic acid compounds, a solubilizer, a humectant, a surfactant, and/or one or more suppository bases. An emollient can also be included in a suppository formulation. An example of a solubilizer is water. An example of a humectant is glycerin. An example of a surfactant is polysorbate 80. Examples of suppository bases are PEG4000, Gelucire 43/01, PEG3350, witepsol W-35®, and saturated fatty acids glycerides C₁₂-C₁₈. An example of an emollient is softisan 378. The one or two polyglutamic acid compounds have molecular weights ranging from 50 kDa to 2000 kDa. For examples, the molecular weight of a polyglutamic acid compound can be 50 kDa, 100 kDa, 150 kDa, 200 kDa, 250 kDa, 300 kDa, 350 kDa, 400 kDa, 450 kDa, 500 kDa, 550 kDa, 600 kDa, 650 kDa, 700 kDa, 750 kDa, 800 kDa, 850 kDa, 900 kDa, 950 kDa, 1000 kDa, 1050 kDa, 1100 kDa, 1150 kDa, 1200 kDa, 1250 kDa, 1300 kDa, 1350 kDa, 1400 kDa, 1450 kDa, 1500 kDa, 1550 kDa, 1600 kDa, 1650 kDa, 1700 kDa, 1750 kDa, 1800 kDa, 1850 kDa, 1900 kDa, 1950 kDa, or 2000 kDa. The weight percentage (% w/w) of each polyglutamic acid compound in any of the compositions described herein is not particularly limited and can range from about 0.01% to about 1%. For example, it can be 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1%, or more, or any value therebetween. The weight percentages (% w/w) of the solubilizer, the humectant, the surfactant, the one or more suppository bases, and the emollient in the suppository formulation are also not particularly limited and can range, respectively, from 1% to 100%, from about 0.1% to 11.4%, from 0.1% to 1.4%, from 0% to 100%, and from 0% to 100%. The amount of the polyglutamic acid compounds, the solubilize, the humectant, the surfactant, the suppository bases and/or the emollient are in a synergistic ratio. As used herein, a “synergistic ratio” refers to a ratio that elicits an unexpectedly superior pharmacological and physiological effect in a subject. An example of the synergistic ratio of the amount of the polyglutamic acid compound to the amount of the solubilizer to the amount of the humectant to the amount of the surfactant, and to the amount of the suppository base is 5:396:50:28:1521.

In certain embodiments, a suppository formulation comprises only one polyglutamic acid compound. In certain embodiments, the compound has a molecular weight of 100 kDa, 700 kDa, or 2000 kDa. In certain embodiments, a suppository formulation contains a solubilizer, a humectant, a surfactant, and a suppository base. In certain embodiments, the solubilizer is water, the humectant is glycerin, the surfactant is polysorbate 80, and the suppository base is saturated fatty acids glycerides C₁₂-C₁₈.

In certain embodiments, a suppository formulation comprises two polyglutamic acid compounds. In certain embodiments, the compounds have molecular weights of 100 kDa and 700 kDa.

In certain embodiments, suppository formulations can be disintegrated between 5-10 minutes, between 10-20 minutes, or between 20-30 minutes. In certain embodiments, a suppository formulation can be administered every two days, every three days, every four days, every five days, every six days, every week, every eight days, every nine days, every ten days, every two weeks, every month, or more to provide fast and long-lasting relief of the symptoms of vaginal dryness and the symptoms associated with menopause.

In certain embodiments, an intravaginal ring formulation described herein can comprise one or more polyglutamic acid compounds and one or more polymers. In certain embodiments, the one or more polyglutamic acid compounds have molecular weights ranging from about 50 kDa to about 2000 kDa, from about 100 kDa to about 2000 kDa, from about 100 kDa to about 700 kDa, or from about 700 kDa to about 2000 kDa. For examples, the molecular weight of a polyglutamic acid compound can be about 50 kDa, 100 kDa, 150 kDa, 200 kDa, 250 kDa, 300 kDa, 350 kDa, 400 kDa, 450 kDa, 500 kDa, 550 kDa, 600 kDa, 650 kDa, 700 kDa, 750 kDa, 800 kDa, 850 kDa, 900 kDa, 950 kDa, 1000 kDa, 1050 kDa, 1100 kDa, 1150 kDa, 1200 kDa, 1250 kDa, 1300 kDa, 1350 kDa, 1400 kDa, 1450 kDa, 1500 kDa, 1550 kDa, 1600 kDa, 1650 kDa, 1700 kDa, 1750 kDa, 1800 kDa, 1850 kDa, 1900 kDa, 1950 kDa, or 2000 kDa and ranges and values therebetween. In certain embodiments, the weight percentage (% w/w) of each polyglutamic acid compound in an intravaginal ring formulation described herein can range from about 1 wt % to about 50 wt %, from about 5 wt % to about 30 wt %, from about 7 wt % to about 25 wt %. For example, the weight percentage (% w/w) of each polyglutamic acid compound in an intravaginal ring formulation can be 1 wt %, 2 wt %, 3 wt %, 4 wt %, 5 wt %, 6 wt %, 7 wt %, 7.5 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 22.5 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, or any value therebetween. In certain embodiments, the total weight percentage (% w/w) of polyglutamic acid compounds in an intravaginal ring formulation described herein can range from 0.01 wt % to about 90 wt %, from about 1 wt % to about 80 wt %, from about 5 wt % to about 50 wt %, from about 10 wt % to about 50 wt %, from about 15 wt % to about 40 wt %, and from about 25 wt % to about 40 wt %. For example, it can be about 0.01 wt %, 1 wt %, 2 wt %, 3 wt %, 4 wt %, 5 wt %, 6 wt %, 7 wt %, 8 wt %, 9 wt %, 10 wt %, 11 wt %, 12 wt %, 13 wt %, 14 wt %, 15 wt %, 16 wt %, 17 wt %, 18 wt %, 19 wt %, 20 wt %, 21 wt %, 22 wt %, 23 wt %, 24 wt %, 25 wt %, 26 wt %, 27 wt %, 28 wt %, 29 wt %, 30 wt %, 31 wt %, 32 wt %, 33 wt %, 34 wt %, 35 wt %, 36 wt %, 37 wt %, 38 wt %, 39 wt %, 40 wt %, 41 wt %, 42 wt %, 43 wt %, 44 wt %, 45 wt %, 46 wt %, 47 wt %, 48 wt %, 49 wt %, 50 wt %, 51 wt %, 52 wt %, 53 wt %, 54 wt %, 55 wt %, 56 wt %, 57 wt %, 58 wt %, 59 wt %, 60 wt %, 61 wt %, 62 wt %, 63 wt %, 64 wt %, 65 wt %, 66 wt %, 67 wt %, 68 wt %, 69 wt %, 70 wt %, 71 wt %, 72 wt %, 73 wt %, 74 wt %, 75 wt %, 76 wt %, 77 wt %, 78 wt %, 79 wt %, 80 wt %, or any value therebetween. A polymer can be natural, semisynthetic, or synthetic suitable for use in medical products. Examples of natural and semisynthetic polymers include, but are not limited to, cellulose and its derivatives (e.g., microcrystalline cellulose; alkyl cellulose including such as methylcellulose, ethylcellulose, and propylcellulose; hydroxyethyl cellulose; hydroxypropyl cellulose; hydroxypropyl methyl cellulose; sodium carboxy methyl cellulose; sodium carboxy ethyl cellulose; and sodium carboxy propyl cellulose), pectin, alginates, starch, carrageenans, chitosan, gelatin, gellan gum, and xanthan gum. Examples of synthetic polymers include, but are not limited to, silicone, poloxamers (e.g., poloxamer 407 and poloxamer 188), polyacrylates (e.g., Eudragit®, Kollicoat®, Eudispert®, and Carbopols®), polyvinylpyrrolidone, polyethylene glycol, polyurethane, polyetheramide, polylactide, polyether, polycaprolactone, alkyds, polyhydroxyurethane, and EVA. A polymer can be thermoplastic. Examples of thermoplastic polymers include, but are not limited to, thermoplastic polyurethane and thermoplastic EVA. In certain embodiments, a polymer is thermoplastic polyurethane such as aliphatic/hydrophilic polyether-based thermoplastic polyurethane. Examples of aliphatic/hydrophilic polyether-based thermoplastic polyurethane include, but are not limited to, Lubrizol MPD-447ZA, Lubrizol Pathway PY-PT95AE60, Lubrizol Pathway PY-PT82AE100, Lubrizol Pathway PY-PT83AE100, and Lubrizol Pathway PY-PT72AE. In certain embodiments, a polymer is thermoplastic EVA. In certain embodiments, a thermoplastic EVA can be EVA 40, EVA 33, EVA 28, EVA 18, EVA 12, EVA 9, and any combination of thereof.

In certain embodiments, an intravaginal ring formulation described herein comprises two polyglutamic acid compounds and one or more polymers. The ratio of a first polyglutamic acid compound to a second polyglutamic acid compound can range from about 10:1 to about 1:10, from about 5:1 to 1:5, from about 3:1 to about 1:3, or from about 3:1 to about 1:1 by their weight percentages in the composition described herein. For example, the ratio of a first polyglutamic acid compound to a second polyglutamic acid compound can be about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, or any values in between. In certain embodiments, the two polyglutamic acid compounds have molecular weights selected from about 100 kDa, 700 kDa, and 2000 kDa. In certain embodiments, a first polyglutamic acid compound has a molecular weight of about 100 kDa and a second polyglutamic acid compound has a molecular weight of about 700 kDa. In certain embodiments, a first polyglutamic acid compound having a molecular weight of about 100 kDa and a second polyglutamic acid compound having a molecular weight of about 700 kDa are provided in a ratio of about 3:1 by their weight percentages in an intravaginal ring formulation described herein. In certain embodiments, the total weight percentage (% w/w) of the two polyglutamic acid compounds in an intravaginal ring formulation can range from about 0.01 wt % to about 90 wt %, from about 1 wt % to about 80 wt %, from about 5 wt % to about 50 wt %, from about 10 wt % to about 50 wt %, from about 25 wt % to about 40 wt %, and from about 15 wt % to about 40 wt %. In certain embodiments, the total weight percentage (% w/w) of the two polyglutamic acid compounds in an intravaginal ring formulation can be about 30 wt %. In certain embodiments, an intravaginal ring formulation described herein can further comprise hyaluronic acid. In certain embodiments, the one or more polymers can be thermoplastic polyurethanes, thermoplastic EVAs, or any combinations thereof. In certain embodiments, the one or more polymers can be thermoplastic EVAs. In certain embodiments, a thermoplastic EVA can be EVA 40, EVA 33, EVA 28, EVA 18, EVA 12, EVA 9, or any combinations thereof. In certain embodiments, the EVAs are selected from the group consisting of EVA 40, EVA 33, EVA 28, EVA 18, EVA 12, or any combinations thereof.

In certain embodiments, an intravaginal ring formulation described herein can be a sustained-release formulation. In certain embodiments, a sustained-released intravaginal ring formulation can be obtained by mixing one or more polymers with one or more polyglutamic acid compounds to form a ring where the one or more polyglutamic acid compounds are dispersed homogeneously in a polymeric matrix formed by the one or more polymers (“intravaginal matrix ring formulation”; see FIG. 2A). In certain embodiments, a sustained-released intravaginal ring formulation can be obtained by mixing one or more polymers with one or more polyglutamic acid compounds to form one or more cores which can be encapsulated by/inserted into an outer polymer membrane sheath (“intravaginal core sheath ring formulation”; see FIGS. 2B-2E). When more than one core is present, the cores can be the same or different. For example, cores can be made from the same polymer(s) and same polyglutamic acid compound(s), cores can be made from the same polymer(s) but different polyglutamic acid compound(s), or cores can be made from different polymer(s) but the same polyglutamic acid compound(s), or the number of polymers/polyglutamic acid compounds in cores are different. In certain embodiments, an outer polymer membrane sheath has one or more holes into which one or more cores can be inserted (see FIG. 2D). In certain embodiments, an intravaginal ring formulation further comprises an inner central core where the one or more cores are placed between the sheath and the inner central core (see FIG. 2E). In certain embodiments, a sheath can be made of silicon dioxide, thermoplastic polyurethane, thermoplastic EVA, or biosoluble hydrogel.

In certain embodiments, an intravaginal matrix ring formulation can comprise two polyglutamic acid compounds and one polymer. In certain embodiments, the two polyglutamic acid compounds can have molecular weights of about 100 kDa and 700 kDa. In certain embodiments, the two polyglutamic acid compounds can have a weight ratio ranging from about 10:1 to about 1:10, from about 5:1 to 1:5, from about 3:1 to about 1:3, or from about 3:1 to about 1:1. For example, the ratio of a polyglutamic acid compound having a molecular weight of about 100 kDa to a polyglutamic acid compound having a molecular weight of about 700 kDa can be about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, or any values in between. In certain embodiments, the total weight percentage (% w/w) of the two polyglutamic acid compounds in an intravaginal matrix ring formulation can range from about 0.01 wt % to about 90 wt %, from about 1 wt % to about 80 wt %, from about 5 wt % to about 50 wt %, from about 10 wt % to about 50 wt %, from about 25 wt % to about 40 wt %, and from about 15 wt % to about 40 wt %. In certain embodiments, the total weight percentage (% w/w) of the two polyglutamic acid compounds in an intravaginal matrix formulation can range from about 1 wt % to about 80 wt %, from about 5 wt % to about 50 wt %, and from about 25 wt % to about 40 wt %. In certain embodiments, the total weight percentage (% w/w) of the two polyglutamic acid compounds in an intravaginal matrix formulation can be about 30 wt %. In certain embodiments, the one polymer in an intravaginal matrix ring formulation can be a thermoplastic EVA. In certain embodiments, the thermoplastic EVA can be selected from the group consisting of EVA 40, EVA 33, EVA 28, EVA 18, EVA 12, and EVA 9. In certain embodiments, the EVAs are selected from the group consisting of EVA 40, EVA 33, and EVA 28. In certain embodiments, the one polymer in an intravaginal matrix ring formulation can be an aliphatic/hydrophilic polyether-based thermoplastic polyurethane. In certain embodiments, the aliphatic/hydrophilic polyether-based thermoplastic polyurethane can be selected from the group consisting of include, but are not limited to Lubrizol MPD-447ZA, Lubrizol Pathway PY-PT95AE60, Lubrizol Pathway PY-PT82AE100, Lubrizol Pathway PY-PT83AE100, and Lubrizol Pathway PY-PT72AE.

In certain embodiments, a sustained-released intravaginal ring formulation is an intravaginal core sheath ring formulation comprising one or more cores and an outer polymer membrane sheath (see FIGS. 2B-2E). In certain embodiments, the one or more cores comprise two polyglutamic acid compounds and one polymer. In certain embodiments, the two polyglutamic acid compounds can have molecular weights of about 100 kDa and about 700 kDa. In certain embodiments, the two polyglutamic acid compounds can have a weight ratio ranging from about 10:1 to about 1:10, from about 5:1 to 1:5, from about 3:1 to about 1:3, or from about 3:1 to about 1:1. For example, the ratio of a polyglutamic acid compound having a molecular weight of about 100 kDa to a polyglutamic acid compound having a molecular weight of about 700 kDa can be about 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, or any values in between. In certain embodiments, the total weight percentage (% w/w) of the two polyglutamic acid compounds in a core can range from about 1 wt % to about 80 wt %, from about 5 wt % to about 50 wt %, from about 25 wt % to about 40 wt %, and from about 25 wt % to about 30 wt %. In certain embodiments, the one core polymer can be a thermoplastic EVA. In certain embodiments, the core thermoplastic EVA can be EVA 40, EVA 33, EVA 28, EVA 18, or EVA 12. In certain embodiments, the core thermoplastic EVA can be EVA 28. In certain embodiments, the sheath polymer can be a thermoplastic EVA. In certain embodiments, the sheath thermoplastic EVA can be EVA 40, EVA 33, EVA 28, EVA 18, EVA 12 or EVA 9. In certain embodiments, the sheath thermoplastic EVA can be EVA 18 or EVA 12. The core polymer and the sheath polymer can be identical or different. In certain embodiments, an intravaginal core sheath ring formulation comprises one core (see FIGS. 2B and 2E). In certain embodiments, an intravaginal core sheath ring formulation comprises multiple cores and the sizes of the cores can be identical or different (FIGS. 2C and 2D). In certain embodiments, a sheath can have a thickness ranging from about 30 μm to about 220 μm or from about 50 μm to about 200 μm. For example, a sheath can have a thickness of 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm, 210 μm, 220 μm, or any value in between.

In certain embodiments, the one or more polyglutamic acid compounds in an intravaginal ring formulation described herein may be released at a sustained manner over a predetermined period of time. For example, the one or more polyglutamic acid compounds in an intravaginal ring formulation described herein may be released at a sustained manner over one week, two weeks, three weeks, one month, two months, three months, four months, five months, six months, seven months, eight months, nine months, ten months, eleven months, one year, or any period time in between. In certain embodiments, the polyglutamic acid compounds described herein in an intravaginal ring formulation described herein may be released at a sustained manner over one week, two weeks, three weeks, one month, two months, three months, or any period time in between. In certain embodiments, the polyglutamic acid compounds described herein in an intravaginal ring formulation described herein may be released at a sustained manner over two weeks to four weeks. In certain embodiments, the polyglutamic acid compounds described herein in an intravaginal ring formulation described herein may be released at a sustained manner over one to three months. In certain embodiments, an intravaginal ring formulation may further comprise two or more ingredients which respond to the same vaginal pH differently and thus release polyglutamic acid compounds at different time and/or release rates. In certain embodiments, an intravaginal ring formulation may further comprise two or more ingredients, some of which increase vaginal pH and some of which lower vaginal pH, and one or more carriers that release the polyglutamic acid compounds at different pH levels. For example, ingredients identified herein that increase vaginal pH can be combined with a carrier that release the polyglutamic acid compounds at a high pH level (e.g., over 4.5) and ingredients identified herein that lower vaginal pH can be combined with a carrier that releases the polyglutamic acid compounds at a low pH level (e.g. below 4.5). In certain embodiments, the two or more polyglutamic acid compounds may be released at the same time or different times.

In certain embodiments, an intravaginal ring formulation described herein may release about 1 mg-10 mg, about 2 mg-9 mg, about 3 mg-8 mg, about 2 mg-5 mg, about 4 mg-7 mg, about 1 mg-6 mg, about 5 mg-8 mg, about 1 mg, about 1.5 mg, about 1.6 mg, about 2 mg, about 2.5 mg, about 2.9 mg, about 3 mg, about 4 mg, about 5 mg, about 5.5 mg, about 6 mg, about 7 mg, per day of the one or more polyglutamic acid compounds described herein.

In certain embodiments, an intravaginal ring formulation can be administered every week, every two weeks, every three weeks, every four weeks, every five weeks, every six weeks, or any times in between to provide sustained relief of the symptoms of vaginal dryness and the symptoms associated with menopause. In certain embodiments, an intravaginal ring formulation described herein can be inserted in the vagina every 7 days and remain there without removal for 7 days. In certain embodiments, an intravaginal ring formulation described herein can be inserted in the vagina every two weeks and remain there without removal for two weeks. In certain embodiments, an intravaginal ring formulation described herein can be inserted in the vagina every three weeks and remain there without removal for three weeks. In certain embodiments, an intravaginal ring formulation described herein can be inserted in the vagina every month and remain there without removal for a month. In certain embodiments, an intravaginal ring formulation described herein can be inserted in the vagina every two months and remain there without removal for two months. In certain embodiments, an intravaginal ring formulation described herein can be inserted in the vagina every three months and remain there without removal for three months. In certain embodiments, an intravaginal ring formulation described herein can be inserted in the vagina every four months and remain there without removal for four months.

In certain embodiments, an intravaginal ring described herein has a cross sectional diameter of about 3.0-10.0 mm and an outer diameter of about 40-80 mm. In certain embodiments, an intravaginal ring described herein has a cross sectional diameter of about 4.0-10.0 mm and an outer diameter of about 46-66 mm. In certain embodiments, an intravaginal described herein has a cross sectional diameter of about 4.0-9.0 mm and an outer diameter of about 53-56 mm. In certain embodiments, an intravaginal ring described herein has a cross sectional diameter of about 4.0-5.0 mm and an outer diameter of about 53-54 mm. In certain embodiments, an intravaginal ring described herein has a cross sectional diameter of about 4.1 mm and an outer diameter of about 53.8 mm. In certain embodiments, an intravaginal ring described herein has a cross sectional diameter of about 4.1 mm and an outer diameter of about 53.3 mm. In certain embodiments, an intravaginal ring described herein has a cross sectional diameter of about 4.1 mm and an outer diameter of about 53.9 mm. In certain embodiments, an intravaginal ring described herein has a cross sectional diameter of about 4.1 mm and an outer diameter of about 53.8 mm. In certain embodiments, an intravaginal ring described herein has a cross sectional diameter of about 4.1 mm and an outer diameter of about 53.5 mm. In certain embodiments, an intravaginal ring described herein has a cross sectional diameter of about 4.1 mm and an outer diameter of about 53.6 mm. In certain embodiments, an intravaginal ring described herein has a cross sectional diameter of about 4.2 mm and an outer diameter of about 53.4 mm. In certain embodiments, an intravaginal ring described herein has a cross sectional diameter of about 4.5 mm and an outer diameter of about 53.3 mm. In certain embodiments, an intravaginal ring described herein has a cross sectional diameter of about 4.2 mm and an outer diameter of about 53.40 mm. In certain embodiments, an intravaginal ring described herein has a cross sectional diameter of about 4.0 mm and an outer diameter of about 54.0 mm.

In certain embodiments, an intravaginal ring described herein can have a 20 mm compression force of about 1.5 N-5 N or about 1.5 N-4.5 N. For example, an intravaginal ring described herein can have a 20 mm compression force of about 1.5 N, 1.6 N, 1.7 N, 1.8 N, 1.9 N, 2.0 N, 2.1 N, 2.2 N, 2.3 N, 2.4 N, 2.5 N, 2.6 N, 2.7 N, 2.8 N, 2.9 N, 3.0 N, 3.1 N, 3.2 N, 3.3 N, 3.4 N, 3.5 N, 3.6 N, 3.7 N, 3.8 N, 3.9 N, 4.0 N, 4.1 N, 4.2 N, 4.3 N, 4.4 N, 4.5 N, 4.6 N, 4.7 N, 4.8 N, 4.9 N, 5.0 N or any value in between. In certain embodiments, an intravaginal ring described herein can have a 33.5 mm (75%) compression force ranging from about 2N to about 12 N, from about 2 N to about 8 N or from about 2.2 N to about 6 N. For example, an intravaginal ring described herein can have a 33.5 mm (75%) compression force of about 2.0 N, 2.1 N, 2.2 N, 2.3 N, 2.4 N, 2.5 N, 2.6 N, 2.7 N, 2.8 N, 2.9 N, 3.0 N, 3.1 N, 3.2 N, 3.3 N, 3.4 N, 3.5 N, 3.6 N, 3.7 N, 3.8 N, 3.9 N, 4.0 N, 4.1 N, 4.2 N, 4.3 N, 4.4 N, 4.5 N, 4.6 N, 4.7 N, 4.8 N, 4.9 N, 5.0 N, 5.1 N, 5.2 N, 5.3 N, 5.4 N, 5.5 N, 5.6 N, 5.7 N, 5.8 N, 5.9 N, 6.0 N, 6.1 N, 6.2 N, 6.3 N, 6.4 N, 6.5 N, 6.6 N, 6.7 N, 6.8 N, 6.9 N, 7.0 N, 7.1 N, 7.2 N, 7.3 N, 7.4 N, 7.5 N, 7.6 N, 7.7 N, 7.8 N, 7.9 N, 8.0 N, 8.1 N, 8.2 N, 8.3 N, 8.4 N, 8.5 N, 8.6 N, 8.7 N, 8.8 N, 8.9 N, 9.0 N, 9.1 N, 9.2 N, 9.3 N, 9.4 N, 9.5 N, 9.6 N, 9.7 N, 9.8 N, 9.9 N, 10.0 N, 10.1 N, 10.2 N, 10.3 N, 10.4 N, 10.5 N, 10.6 N, 10.7 N, 10.8 N, 10.9 N, 11.0 N, 11.1 N, 11.2 N, 11.3 N, 11.4 N, 11.5 N, 11.6 N, 11.7 N, 11.8 N, 11.9 N, 12.0 N, or any value in between. In certain embodiments, an intravaginal ring described herein can have a break force of about 50 N-130 N and a break length of about 40 mm-280 mm. For example, an intravaginal ring described herein can have a break force of about 92.4 N and a break length of about 44.8 mm, a break force of about 97.2 N and a break length of about 266.8 mm, a break force of about 95.4 N and a break length of about 215.1 mm, a break force of about 123.2 N and a break length of about 274.3 mm, a break force of about 104.7 N and a break length of about 300 mm, a break force of about 112.1 N and a break length of about 257.7 mm, a break force of about 55.1 N and a break length of about 97.6 mm, a break force of about 78.1 N and a break length of about 103.9 mm, a break force of about 92.4 N and a break length of about 44.8 mm, or a break force of about 98.9 N and a break length of about 83.7 mm. In certain embodiments, an intravaginal ring described herein can have a 200N elongation length of about 216.5 mm or about 31.2 mm. In certain embodiments, an intravaginal ring described herein can have a 182.3N elongation length of about 300 mm. In certain embodiments, an intravaginal ring described herein can have Shore A Hardness of about 60-90, about 64-88 or about 64-80. For example, an intravaginal ring described herein can have Shore A Hardness of about 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90 or any value in between.

While the present invention has been described in some detail for purposes of clarity and understanding, one will appreciate that various changes in form and detail can be made without departing from the true scope of the invention.

### EXAMPLES

### Example 1

In one example, polyglutamic acid (mw: 100 kDa; 5 mg; 0.25% w/w) was mixed with water (396 mg; 19.8% w/w), glycerin (50 mg; 2.5% w/w), polysobrate 80 (28 mg; 1.4% w/w), and saturated fatty acids glycerides C₁₂-C₁₈ (1521 mg; 76.04% w/w) to yield a white suppository. A dissolution study of the suppository was conducted by using a phosphate buffer that has a pH range of about 4.2-4.5, which is within a range of normal vaginal pH (3.8-5.0). Percentages of polyglutamic acid in the buffer at hour 0.5, hour 0.75, hour 1, hour 2, hour 3, hour 6, hour 24, hour 30, and hour 48 were determined. The dissolution data are shown in Table 1 below.

TABLE 1
Dissolution Data

| Time | % Dissolved | | |
|---|---|---|---|
| (hr) | Average (n = 3) | Std Dev | % RSD |
| 0.5 | 67.16 | 13.24 | 19.7 |
| 0.75 | 68.27 | 19.91 | 29.2 |
| 1 | 72.97 | 19.12 | 26.2 |
| 2 | 88.21 | 9.90 | 11.2 |
| 3 | 98.09 | 1.78 | 1.8 |
| 4 | 100.01 | 4.19 | 4.2 |
| 24 | 101.83 | 3.25 | 3.2 |
| 30 | 100.64 | 3.09 | 3.1 |
| 48 | 102.85 | 3.13 | 3.0 |

It was observed that the suppository completely disintegrated within 30 minutes. The data shows that 67% of the polyglutamic acid compound was dissolved in 30 minutes and the polyglutamic acid compound was completely dissolved in the buffer at hour 3. Unexpectedly, a steady dissolution was observed from hour 0.5 to hour 3. Also unexpectedly, 100% of the polyglutamic acid compound remained in the buffer at hour 48, indicating that the compound is stable in a vaginal tissue and therefore, can moisturize the vaginal tissue for more than 2 days once administered. These unexpected results demonstrate that the composition can provide fast and long-lasting relief of the symptoms of vaginal dryness.

### Example 2

The vaginal suppository composition described in example 1 above will be studied to evaluate its short-term stability as compared to a placebo prototype composition. The placebo composition will be identical to the vaginal suppository composition except that the former does not contain any polyglutamic acid compound. Both compositions will be placed on ICH stability at 2-8° C. and 25° C./60% RH conditions for 3 months. At the end of month 1 and month 2, the stability of the vaginal suppository composition will be evaluated by visual appearance and the amount of the polyglutamic acid compound remained in the composition. At the end of month 3, the stability of the vaginal suppository composition will be evaluated by, in addition to visual appearance and the amount of polyglutamic acid remaining in the composition, the amount of the polyglutamic acid compound dissolved. As to the placebo composition, its stability will be evaluated by visual appearance and placebo interference at the end of each month.

### Example 3

An assay will be conducted where an in vitro vaginal tissue test apparatus will be used to test the activity and efficacy of varying polymer lengths of polyglutamic acid compounds. The varying polyglutamic acid compounds (i.e., with different n values) will be applied to the test apparatus, and their ability to penetrate into the various tissue layers of the vaginal wall will be correlated with their n value (i.e., the number of monomers in the polyglutamic acid compound), and the relative effect on vaginal moisture. In addition, compositions having varying concentrations of polyglutamic acid compounds with different polymer chain lengths (i.e., different n values) will be tested to ascertain the effect on vaginal moisture and penetration into vaginal tissue layers.

### Example 4

Certain compositions described herein will be tested for their effects on the vaginal epithelium in a postmenopausal rat model.

Thirty female Wistar/Sprague rats (eight-week-old) will be purchased from the Firat University Experimental Animal Center. All rats will be individually housed under clean conditions with controlled temperature, humidity, and light (12-h light-dark cycle) and provided a standard commercial diet and water ad libitum. All experimental procedures will follow the guidelines established by the Use of Laboratory Animals and will be approved by the Animal Care and Use Committee of Firat University.

Rats that exhibited regular 4-days oestrous cycle will be chosen for the experiment. The rats will be divided into seven groups as shown in Table 2. After one adaptation period, rats will be ovariectomized (OVX) or given a sham operation (control) under anesthesia. The same weight of adipose tissue will be removed from rats in the control group. After the surgery, the vaginal smears will be assessed daily. Only rats whose vaginal smears that have exhibited regular 4-days cycles (before and after sham/OVX) and rats that exhibited constant leukocytes (after OVX) will be used (10 rats for each group). The rest of rats will be dosed intra-vaginally or orally with the same volume of placebo (vehicle) gel as rats that received a composition. The body weights of rats will be measured weekly.

TABLE 2
Study groups

| | | | Animal dose (per rat/day) |
|---|---|---|---|
| Groups | Source | HED | (n = 10 per arm)* |
| 1 | Saline | — | — |
| 2 | OVX | — | |
| 3 | OVX+ | 5 mg | 0.51 mg/kg BW |
| 4 | OVX+ | 50 mg | 5.14 mg/kg BW |
| 5 | OVX+ | 500 mg | 51.42 mg/kg BW |
| 6 | OVX+ | 5000 mg | 514.16 mg/kg BW |
| 7 | OVX+ | 50 g | 0.52 g/kg BW |

*Doses of products were converted by allometric scaling, which takes into account differences in body surface area to calculate equivalent doses for rat by the following calculation, assuming an 60 kg human: RD = HED(g)/60 kg*6.17 (For example: (50 mg/60)*6.17 = 5.14 mg/kg BW); where RD is the rat dosage of products, HED is the human equivalent dosage of products, and 6.17 is the conversion factor to convert a human dosage to a rat dosage

On day 15, the treatment effect of each composition for vaginal atrophy will be determined by a vaginal smear obtained from each rat, with a histological examination of a vaginal tissue. Vaginal smears will be fixed and detected, the expression of leukocyte esterase, β-glucuronidase, and coagulase will be used to determine the effect of each composition on vaginal microecosystem. Then all rats will be sacrificed by cervical dislocation and their vagina and uterus will be removed. The wet weight of each uterus will be determined once they will be carefully cleaned of any adherent tissue. To minimize variability due to dissection, all uteri will be dissected above the cervix at the same distance. The vaginal tissue will be then removed and will be cut into 1 mm×1 mm×2 mm sections, stained with 2.5% glutaraldehyde. Some vaginal samples will be taken and processed for histological examination by standard procedures (4-5 μm sections stained with haematoxylin plus eosin).

The repair capacity of each composition will be detected by Western blot. Vaginal VEGF, p-AKT, COX-2, ERα, and ERβ will be detected by Western blot method. For Western blot, proteins will be separated in 10% polyacrylamide gel, electrotransferred topolyvinylidene fluoride membranes, and the bands will be blocked in 5% dried milk for 1 hour at room temperature and incubated with different primary antibodies (1:1000). The incubation will be carried out in PBS supplemented with 5% skim milk powder. After washing with Tris-Buffered Saline (TBS) containing 0.1% Tween-20 (TBS-T), the membranes will be further incubated with an anti-rabbit IgG antibody conjugated with horseradish peroxidase at room temperature for 1 hour. After washing with TBS-T, antigen-antibody complex will be visualized by using an enhanced-chemiluminescence-detection kit.

### Example 5

Intravaginal matrix ring formulations were prepared by the following steps: (1) cryo-milling a polymer to a powder in liquid nitrogen using a mill (e.g., Retsch ZM200 Mill) and removing residual moisture if necessary using a dryer (e.g., Dri-Air NAFM Polymer Dryer); (2) dry-blending the resultant polymer powder with polyglutamic acid compounds using a mixer (e.g., Glenn Mills T2F Turbula Mixer); (3) extruding the resultant powder blend using a twin-screw extruder (e.g., Leistritz ZSE 18 Twin-Screw Extruder) followed by pelletizing using a pelletizer (e.g., Bay Plastics BT25 Pelletizer) to give polyglutamic-acid-compounds-loaded polymer pellets; and (4) injecting the pellets to a molder (e.g., BabyPlast 610P Injection Molder) to form rings.

Seven intravaginal matrix ring formulations were prepared. Each ring comprises about 30 wt % of total amounts of two polyglutamic acid compounds having molecular weights of about 100 kDa and about 700 kDa at a ratio of 3:1 by weight. The polymer in each ring is shown in Table 3. The physical properties of the rings compared to NuvaRing are also shown in Table 3.

TABLE 3

| | Dimensions | | 33.5 mm | | |
|---|---|---|---|---|---|
| | Cross | 20 mm | (75%) | | |
| | Section/Outer | Compression | Compression | Tensile | Shore A |
| Sample | Diameter | Force | Force | Robustness | Hardness |
| NuvaRing | 4.0 mm/53.7 mm | 2.605N | 5.232N | N/A | 76 |
| MPD- | 4.1 mm/53.8 mm | 5.691N | 7.690N | Break Force = | 76 |
| 447ZA | | | | 98.9N | |
| w/30% PGA | | | | Break Length = | |
| (Formulation 1) | | | | 83.7 mm | |
| Pathway PY- | 4.1 mm/53.3 mm | 1.574N | 2.388N | 200N | 71 |
| PT72AE | | | | Elongation | |
| w/30% PGA | | | | length = | |
| (Formulation 2) | | | | 216.5 mm* | |
| Pathway PY- | 4.1 mm/53.9 mm | 8.270N | 11.629N | 200N | 88 |
| PT95AE 60 | | | | Elongation | |
| w/30% PGA | | | | length = | |
| (Formulation 3) | | | | 31.2 mm* | |
| Pathway PY- | 4.1 mm/53.8 mm | 4.314N | 6.644N | 182.3N | 84 |
| PT83AE 100 | | | | Elongation | |
| w/30% PGA | | | | length = | |
| (Formulation 4) | | | | 300 mm | |
| EVA 28 | 4.1 mm/53.8 mm | 4.344N | 5.929N | Break Force = | 80 |
| (28% VA) | | | | 92.4N | |
| w/30% PGA | | | | Break Length = | |
| (Formulation 5) | | | | 44.8 mm | |
| EVA 33 | 4.1 mm/53.9 mm | 3.225N | 4.264N | Break Force = | 76 |
| (33% VA) | | | | 78.1N | |
| w/30% PGA | | | | Break Length = | |
| (Formulation 6) | | | | 103.9 mm | |
| EVA 40 | 4.1 mm/53.5 mm | 1.530N | 2.232N | Break Force = | 64 |
| (40% VA) | | | | 55.1N | |
| w/30% PGA | | | | Break Length = | |
| (Formulation 7) | | | | 97.6 mm | |

*Rings reached maximum force of pull without breaking or reaching full length
**Rings stretched to maximum length of equipment without reaching maximum force of cell

In-vitro elution study of the ring formulations was conducted.

Before the elution study, a solution to be used in the study was determined by a saturation solubility study where saturation solubility of two polyglutamic acid compounds having molecular weights of about 700 kDa and about 100 kDa was assessed individually in four solutions, i.e., water, pH 4.5 acetate buffer, pH 6.8 phosphate buffer, and 0.1N HCl. Each polyglutamic acid compound was added to each solution until the polyglutamic acid compound remained undissolved visibly, or until about 100 mg was added to a 5 mL solution. Then the resultant solutions were placed in an incubator shaker at 37° C. and 60 rpm overnight. It was observed that no visible polyglutamic acid compounds remained. Solubility requirement to maintain sink conditions was estimated to be about 16.9 mg/mL for the polyglutamic acid compound having a molecular weight of about 100 kDa and 5.6 mg/mL for the polyglutamic acid compound having a molecular weight of about 700 kDa based on 30% loading, about 2.5 g weight of a ring, 100 mL dissolution volume and a sink safety factor of 3 using the following formula:

$$2.5 g * 1000 mg / g * 30 \% / 100 mL * 3 = 22.5 mg / ml$$

polyglutamic acid compound 100 kDa: 22.5 mg/ml*75%=16.9 mg/mL
polyglutamic acid compound 700 kDa: 22.5 mg/mL*25%=5.6 mg/mL.

TABLE 4
Saturation Solubility for polyglutamic
acid compounds having molecular weights
of about 100 kDa and 700 kDa

| | 700 kDa | 100 kDa ) |
|---|---|---|
| Solution | (mg/mL) | (mg/mL |
| water | ≥23 mg/mL | ≥20 mg/mL |
| pH 4.5 acetate buffer | ≥26 mg/mL | ≥20 mg/mL |
| pH 6.8 phosphate buffer | ≥20 mg/mL | ≥21 mg/mL |
| 0.1N HCl | ≥17 mg/mL | ≥21 mg/mL |

As shown in Table 4, sink conditions were achieved with all solutions tested. Water was selected as the solution for the in-vitro elution study because it is easier to prepare.

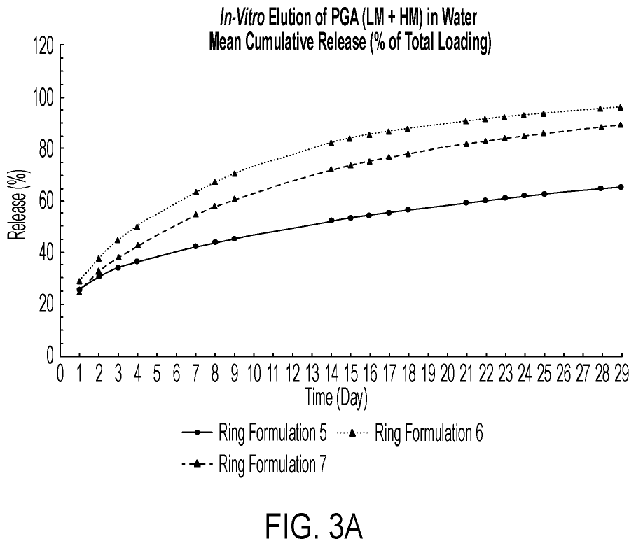

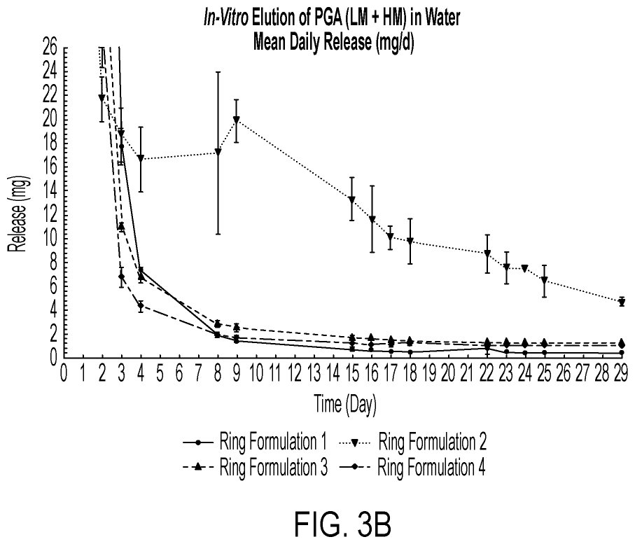

In-vitro elution study was conducted by the following procedure: each ring was placed in water (100 mL) in a glass jar, and then the glass jar was shaken at 60 rpm at 37° C. Mean cumulative release percentages of both polyglutamic acid compounds were determined each day (i.e., timepoints) over a period of 29 days. After each timepoint, the water was replaced with fresh water (100 mL). As shown in FIG. 3A, Formulations 5-7 unexpectedly released the two polyglutamic acid compounds steadily from day 1 to day 29. As shown in FIG. 3B, Formulations 1~4 released the two polyglutamic acid compounds steadily from day 2 to day 29.

### Example 6

Five intravaginal core sheath ring formulations were prepared by the following steps: (1) cryo-milling a polymer to a powder in liquid nitrogen using a mill (e.g., Retsch ZM200 Mill) and removing residual moisture if necessary using a dryer (e.g., Dri-Air NAFM Polymer Dryer); (2) dry-blending the resultant polymer powder with polyglutamic acid compounds using a mixer (e.g., Glenn Mills T2F Turbula Mixer); (3) extruding the resultant powder blend using a twin-screw extruder (e.g., Leistritz ZSE 18 Twin-Screw Extruder) followed by pelletizing using a pelletizer (e.g., Bay Plastics BT25 Pelletizer) to give polyglutamic-acid-compounds-loaded polymer pellets; (4) co-extruding core material and sheath material in single-screw extruders (e.g., Brabender ¾ inch single screw extruders) through a dual-layer crosshead die (e.g., Vortex Model 101 Dual-Layer Crosshead) to form strands; (5) cutting strands using a cutter (e.g., Conair MedLine Puller/Cutter); and (6) thermally welding ends of the strands together via induction energy to form rings using a ring bonder (e.g., PlasticWeld Systems HPS Ring Bonder). Each formulation comprises about 30 wt % of total amounts of two polyglutamic acid compounds having molecular weights of about 100 kDa and about 700 kDa at a ratio of 3:1 by weight. The formulation details are set forth in Table 5 and their physical properties compared to NuvaRing and Formulation 5 are listed in Table 6 below.

TABLE 5

| | | | Sheath |
|---|---|---|---|
| | | Sheath | Thickness |
| Formulation | Core component | Polymer | (μm) |
| 8 | EVA 28 w/30% PGAs | EVA 18 | 50 |
| 9 | EVA 28 + w/30% PGAs | EVA 18 | 100 |
| 10 | EVA 28 + w/30% PGAs | EVA 18 | 200 |
| 11 | EVA 28 + w/30% PGAs | EVA 12 | 100 |
| 12 | EVA 28 + w/30% PGAs | EVA 12 | 200 |

TABLE 6

| | Dimensions | 20 mm | 33.5 mm (75%) | | |
|---|---|---|---|---|---|
| | Cross Section/ | Compression | Compression | Tensile | Shore A |
| Sample | Outer Diameter | Force | Force | Robustness | Hardness |
| NuvaRing | 4.0 mm/ | 2.605N | 5.232N | N/A | 76 |
| | 53.7 mm | | | | |
| EVA 28 | 4.1 mm/ | 4.344N | 5.929N | Break | 80 |
| w/30% PGA | 53.8 mm | | | Force = | |
| (Formulation 5) | | | | 92.4N | |
| | | | | Break | |
| | | | | Length | |
| | | | | 44.8 mm | |
| Formulation 8 | 4.1 mm/ | 2.409N | 3.522N | Break | 76 |
| | 53.3 mm | | | Force = | |
| | | | | 97.2N | |
| | | | | Break | |
| | | | | Length | |
| | | | | 266.8 mm | |
| Formulation 9 | 4.1 mm/ | 2.677N | 3.728N | Break | 73 |
| | 53.9 mm | | | Force = | |
| | | | | 95.4N | |
| | | | | Break | |
| | | | | Length = | |
| | | | | 215.1 mm | |
| Formulation 10 | 4.1 mm/ | 2.515N | 3.655N | Break | 69 |
| | 53.6 mm | | | Force = | |
| | | | | 123.2N | |
| | | | | Break | |
| | | | | Length = | |
| | | | | 274.3 mm | |
| Formulation 11 | 4.2 mm/ | 2.623N | 3.744N | Break | 73 |
| | 53.4 mm | | | Force = | |
| | | | | 104.7N | |
| | | | | Break | |
| | | | | Length = | |
| | | | | 300 mm | |
| Formulation 12 | 4.5 mm/ | 2.724N | 3.87N | Break | 72 |
| | 53.3 mm | | | Force = | |
| | | | | 112.1N | |
| | | | | Break | |
| | | | | Length = | |
| | | | | 257.7 mm | |

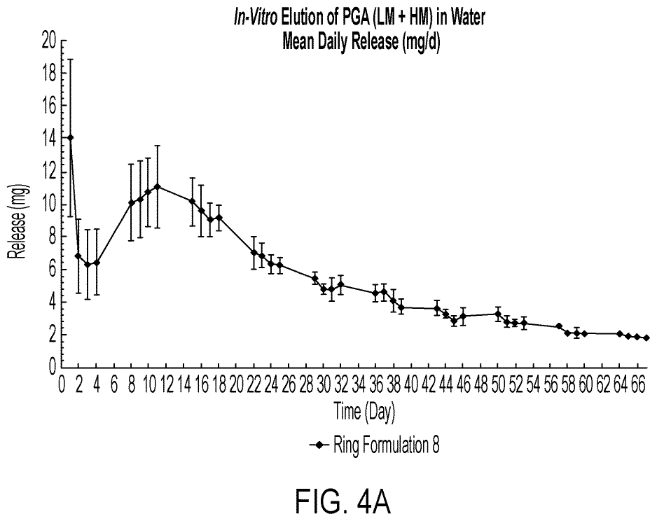

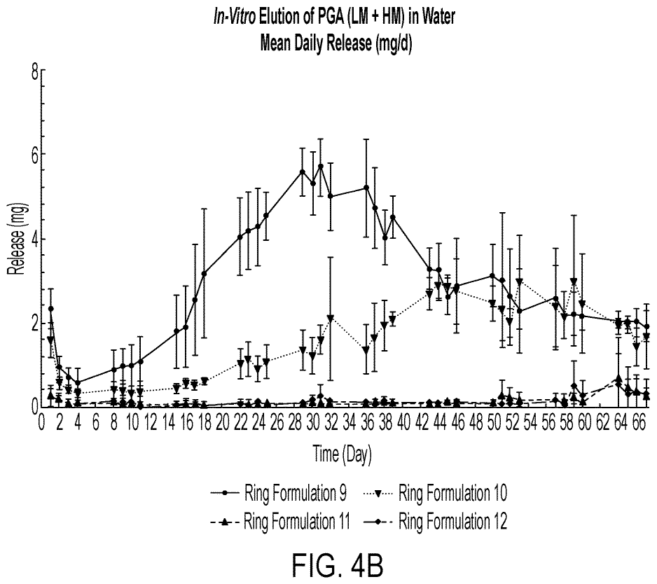

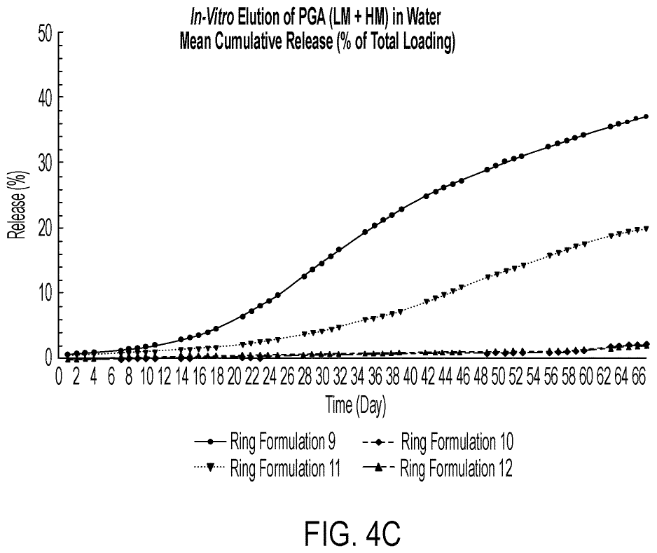

In-vitro elution study of Formulations 8-12 were conducted using the procedure described above. The results are shown in FIGS. 4A-4C.

FIG. 4A shows that Formulation 8 sustainedly released the two polyglutamic acid compounds at a daily total average amount of about 5.5 mg/day over 67 days. FIG. 4B shows that Formulations 9 and 11 sustainedly released the two polyglutamic acid compounds at a daily total average amount of about 2.9 mg/day and 1.6 mg/day from day 1 to day 67. FIGS. 4B and 4C show Formulations 9 and 11 sustainedly released the two polyglutamic acid compounds, unexpectedly much better than Formulations 6 and 8. FIG. 4C also shows that Formulation 9 at day 67 released about 40% of the two polyglutamic acid compounds and Formulation 11 at day 67 released about 20% of the two polyglutamic acid compounds, indicating that release of the two polyglutamic acid compounds from Formulations 9 and 11 can go beyond 90 days, which is unexpected.

1. An intravaginal ring formulation for treating vaginal dryness comprising one or more polyglutamic acid compounds and one or more polymers.

2. The intravaginal ring formulation of claim 1, wherein the one or more polyglutamic acid compounds comprise a first polyglutamic acid compound and a second polyglutamic acid compound.

3. The intravaginal ring formulation of claim 2 further comprising a third polyglutamic acid compound.

4. The intravaginal ring formulation of claim 2, wherein the first polyglutamic acid compound has an n value that is less than an n value of the second polyglutamic acid compound, wherein the n value is the number of the glutamic acid monomers linked together to form each polyglutamic acid compound.

5. The intravaginal ring formulation of claim 3, wherein the first polyglutamic acid compound has an n value that is less than an n value of the second polyglutamic acid compound, and the third polyglutamic acid compound has an n value that is greater than the n value of the second polyglutamic acid compound, wherein the n value is the number of the glutamic acid monomers linked together to form each polyglutamic acid compound.

6. The intravaginal ring formulation of claim 2, wherein the amount of the first polyglutamic acid compound and amount of the second polyglutamic acid compound are provided in a ratio ranging from 10:1 to 1:10 by their weight percentages in the formulation.

7. The intravaginal ring formulation of claim 2, wherein the first polyglutamic acid compound has a molecular weight of about 100 kDa and the second polyglutamic acid compound has a molecular weight of about 700 kDa.

8. The intravaginal ring formulation of claim 1, wherein the one or more polymers are thermoplastic polyurethanes, thermoplastic EVAs, or any combinations thereof.

9. The intravaginal ring formulation of claim 1 is provided in a sustained-release form.

10. The intravaginal ring formulation of claim 9, wherein the formulation is an intravaginal matrix ring formulation.

11. The intravaginal ring formulation of claim 9, wherein the formulation is an intravaginal core sheath ring formulation.

12. The intravaginal ring formulation of claim 10, wherein the formulation comprises two polyglutamic acid compounds and one polymer.

13. The intravaginal ring formulation of claim 12, wherein the two polyglutamic acid compounds have molecular weights of about 100 kDa and about 700 kDa.

14. The intravaginal ring formulation of claim 12, wherein the one polymer is selected from the group consisting of EVA 40, EVA 33, EVA 28, and EVA 12.

15. The intravaginal ring formulation of claim 11, wherein the formulation comprises two polyglutamic acid compounds and two polymers.

16. The intravaginal ring formulation of claim 15, wherein the two polyglutamic acid compounds have molecular weights of about 100 kDa and about 700 kDa.

17. The intravaginal ring formulation of claim 15, wherein the two polyglutamic acid compounds and the one polymer forms a core, wherein the other polymer forms a sheath, and wherein the core is encapsulated by or inserted into the sheath.

18. The intravaginal ring formulation of claim 17, wherein the core polymer and the sheath polymer are independently selected from the group consisting of EVA 40, EVA 33, EVA 28, and EVA 12.

19. The composition of claim 1, wherein the intravaginal ring has a cross sectional diameter of about 4.0-9.0 mm and an outer diameter of about 54-56 mm.

20. A method of treating vaginal dryness, maintaining a healthy level of vaginal moisture, and/or treating, preventing, and/or ameliorating one or more symptoms of menopause, comprising administering an intravaginal ring formulation to a subject's vaginal cavity, wherein the intravaginal ring formulation comprises one or two polyglutamic acid compounds and one or more polymers.

21. The method of claim 20, wherein the intravaginal ring formulation comprises a first polyglutamic acid compound and a second polyglutamic acid compound.

22. The method of claim 21, wherein the amount of the first polyglutamic acid compound and amount of the second polyglutamic acid compound are provided in a ratio ranging from 10:1 to 1:10 by their weight percentages in the formulation.

23. The method of claim 20, wherein the intravaginal ring formulation is provided in a sustained-release form.

24. The method of claim 20, wherein the intravaginal ring has a cross sectional diameter of about 4.0-9 mm and an outer diameter of about 54-56 mm.

25. The method of claim 20, wherein the one or more polymers are thermoplastic polyurethanes, thermoplastic EVAs, or any combinations thereof.

26. The method of claim 23, wherein the formulation is an intravaginal matrix ring formulation.

27. The method of claim 23, wherein the formulation is an intravaginal core sheath ring formulation.

28. The method of claim 26, wherein the formulation comprises two polyglutamic acid compounds and one polymer.

29. The method of claim 28, wherein the two polyglutamic acid compounds have molecular weights of about 100 kDa and about 700 kDa.

30. The method of claim 28, wherein the one polymer is selected from the group consisting of EVA 40, EVA 33, EVA 28, and EVA 12.

31. The method of claim 27, wherein the formulation comprises two polyglutamic acid compounds and two polymers.

32. The method of claim 31, wherein the two polyglutamic acid compounds have molecular weights of about 100 kDa and 700 kDa.

33. The method of claim 31, wherein the two polyglutamic acid compounds and the one polymer forms a core, wherein the other polymer forms a sheath, and wherein the core is encapsulated by or inserted into the sheath.

34. The method of claim 33, wherein the core polymer and the sheath polymer are selected from the group consisting of EVA 40, EVA 33, EVA 28, and EVA 12.

35. The method of claim 20, wherein the symptoms are vaginal dryness, vaginal itching, vaginal burning, and/or pain associated with sexual activity.

36. The method of claim 20, wherein the pain is a result of sexual intercourse.
